(19) United States
(12) Patent Application Publication
(10) Pub. No.: US 2025/0011860 A1
(43) Pub. Date: Jan. 9, 2025

(54) MOLECULAR INDEXING METHODS TO INCREASE THROUGHPUT OF NGS-BASED MULTIPLEX IMMUNOASSAYS

(71) Applicant: GUARDANT HEALTH, INC., Palo Alto, CA (US)

(72) Inventor: Andrew KENNEDY, San Diego, CA (US)

(21) Appl. No.: 18/766,276

(22) Filed: Jul. 8, 2024

Related U.S. Application Data
(60) Provisional application No. 63/512,487, filed on Jul. 7, 2023.

(51) Int. Cl. C12Q 1/6869; C12Q 1/6816; C12Q 1/686; C12Q 1/6876

(52) U.S. Cl. CPC C12Q 1/6869; C12Q 1/6816; C12Q 1/686; C12Q 1/6876

(57) ABSTRACT

Methods are disclosed for detecting and identifying target molecules, such as proteins, in a plurality of biological samples using high throughput methods. A molecular indexing approach is incorporated into next generation sequencing-based immunoassay workflows that allows for pooling of probe panels targeting molecules of interest to be mixed prior to sequencing, thus increasing throughput and reducing costs.

### CROSS-REFERENCE TO RELATED APPLICATIONS

This application claims benefit of U.S. Provisional Patent Application No. 63/512,487 filed Jul. 7, 2023, which is incorporated by reference herein in its entirety.

### FIELD

Embodiments of the present disclosure relate generally to methods of screening, detecting, and/or determining therapies for diseases such as cancer, and more specifically to identifying target molecules, such as protein biomarkers, and tagging them with unique sequences or barcodes for high throughput sequencing.

### BACKGROUND

It has been previously shown that the presence of cancer, either due to secretion of proteins from the tumor itself, or secretions from other cells in the tumor microenvironment (such as immune cells, stromal cells, etc.) is associated with differing levels of certain marker proteins. Using such proteins to detect early-stage cancers shows promise, but such methods are not readily adaptable to high throughput screening methods. Thus, there is a need for methods to increase the number of protein samples that can be detected and processed for identifying disease states such as cancer.

### BRIEF SUMMARY

The present disclosure addresses these deficiencies by providing a molecular indexing approach for multiplex immunoassays that is capable of efficiently processing large populations of immunoassay targets.

In an aspect, the present disclosure provide a method for detecting a polypeptide in a plurality of biological samples, comprising: (a) contacting a first sub-sample of each biological sample with a first panel of probes and contacting a second sub-sample of each biological sample with a second panel of probes, wherein each probe comprises a polypeptide binding domain that specifically recognizes a polypeptide target and a polynucleotide domain that includes at least one target barcode and at least one hybridization domain; (b) enabling the probes to bind to said polypeptide target; (c) enabling the at least one hybridization domain to hybridize and form a polynucleotide duplex; (d) amplifying said polynucleotide duplex, comprising said target barcodes, and generating a population of polynucleotide tags; (e) modifying said polynucleotide tags by: 1) incubating said population of polynucleotide tags with extension primers capable of hybridizing to the polynucleotide tags and of incorporating at least one additional barcode into said polynucleotide tag, 2) amplifying said population of polynucleotide tags to incorporate the at least one additional barcode; (f) repeating said modifying; and (g) identifying the polypeptide using said modified polynucleotide tag, wherein the at least one additional barcode of step (e) is a sample barcode and the at least one additional barcode of step (f) is a panel barcode.

In an aspect, the present disclosure provide for a method for detecting a polypeptide in a plurality of biological samples, comprising: (a) contacting a first sub-sample of each biological sample with a first panel of probes and contacting a second sub-sample of each biological sample with a second panel of probes, wherein each probe comprises a polypeptide binding domain that specifically recognizes a polypeptide target and a polynucleotide domain that includes at least one target barcode and at least one hybridization domain; (b) enabling the probes to bind to said polypeptide target; (c) enabling the at least one hybridization domain to hybridize and form a polynucleotide duplex; (d) amplifying said polynucleotide duplex, comprising said target barcodes, and generating a population of polynucleotide tags; (e) modifying said polynucleotide tags by: 1) incubating said population of polynucleotide tags with extension primers capable of hybridizing to the polynucleotide tags and of incorporating at least one additional barcode into said polynucleotide tag, 2) amplifying said population of polynucleotide tags to incorporate the at least one additional barcode; (f) pooling the modified polynucleotide tags derived from the first sub-samples to generate a first pool and pooling the modified polynucleotide tags derived from the second sub-samples to generate a second pool, and repeating said modifying for each pool; and (g) identifying the polypeptide using said modified polynucleotide tag, wherein the at least one additional barcode of step (e) is a sample barcode and the at least one additional barcode of step (f) is a panel barcode.

In an aspect, the method comprises: contacting a biological sample with at least one set of probes, wherein each probe comprises a polypeptide binding domain that specifically recognizes a polypeptide target and a polynucleotide domain that includes at least one unique barcode and at least one hybridization domain; enabling each at least one set of probes to bind to each said polypeptide target; enabling the at least one hybridization domain to hybridize and form a polynucleotide duplex; amplifying said polynucleotide duplex, comprising said unique barcodes, and generating a population of polynucleotide tags; modifying said polynucleotide tags by: incubating said population of polynucleotide tags with extension primers capable of hybridizing and catalyzing extension of the polynucleotide tags and of incorporating at least one additional unique barcode into said polynucleotide tag; amplifying said population of polynucleotide tags to incorporate the at least one additional unique barcode; and identifying the polypeptide using said modified polynucleotide tag.

In some embodiments, the method includes pooling together a population of modified polynucleotide tags prior to said identifying, wherein the pooled population includes modified polynucleotide tags from different biological samples, from different polypeptide panels, or from different experimental conditions.

In some embodiments, the method includes repeating the modifying process more than once prior to the identifying process.

In some embodiments, a first probe comprises a forward nucleic acid and a second probe comprises a complementary reverse nucleic acid sequence that enables dual recognition of a target molecule in a sample. In some embodiments, the at least one hybridization domain on said polynucleotide domain of a probe is complementary to at least one hybridization domain on a polynucleotide domain of another probe. In some embodiments, the at least one hybridization domain on said polynucleotide domain is complementary to at least one hybridization domain on the same polynucleotide. In some embodiments the probes are antibodies.

In some embodiments, the population of polynucleotide tags is divided into separate aliquots prior to the amplifying of the polynucleotide duplex.

In some embodiments, the amplifying comprises a polymerase chain reaction or a quantitative polymerase chain reaction.

In an aspect, the method comprises: contacting a biological sample with at least one set of probes, wherein each probe comprises a polypeptide binding domain that specifically recognizes a polypeptide target and a polynucleotide domain that includes at least one unique barcode and at least one hybridization domain; enabling each at least one set of probes to bind to each said polypeptide target; enabling the at least one hybridization domain to hybridize and form a polynucleotide duplex; amplifying said polynucleotide duplex, comprising said unique barcodes, and generating a population of polynucleotide tags; modifying said polynucleotide tags by: incubating said population of polynucleotide tags with extension primers capable of hybridizing and catalyzing extension of the polynucleotide tags and of incorporating at least one additional unique barcode into said polynucleotide tag; amplifying said population of polynucleotide tags to incorporate the at least one additional unique barcode; and identifying the polypeptide using said modified polynucleotide tag.

In some embodiments, the molecule in a biological sample is selected from the group consisting of proteins, polynucleotides, and protein complexes.

In some embodiments, the method includes pooling together a population of modified polynucleotide tags prior to said identifying, wherein said pooled population includes modified polynucleotide tags from different biological samples, from different polypeptide panels, or from different experimental conditions.

In some embodiments, the modifying process is repeated more than once prior to the identifying process.

In some embodiments, the at least one hybridization domain on the polynucleotide domain is complementary to at least one hybridization domain on the same polynucleotide.

In some embodiments, the population of polynucleotide tags is divided into separate aliquots prior to said amplifying of said polynucleotide duplex.

In some embodiments, the amplifying comprises a polymerase chain reaction or a quantitative polymerase chain reaction.

In an aspect, the method of detecting a molecule in a biological sample, comprises: contacting said biological sample with at least one probe, wherein each probe comprises a polypeptide binding domain that specifically recognizes a target on said polypeptide and a polynucleotide domain that includes at least one unique barcode and at least one hybridization domain; enabling each at least one probe to bind to each said polypeptide target; enabling the at least one hybridization domain to hybridize and form a polynucleotide duplex; amplifying said polynucleotide duplex, comprising said unique barcodes, and generating a population of polynucleotide tags; modifying said polynucleotide tags by: incubating said population of polynucleotide tags with extension primers capable of hybridizing and catalyzing extension of the polynucleotide tags and of incorporating at least one additional unique barcode into said polynucleotide tag; amplifying said population of polynucleotide tags to incorporate the at least one additional unique barcode; and identifying the polypeptide using said modified polynucleotide tag.

In some embodiments, the molecule in a biological sample is selected from the group consisting of: proteins, polynucleotides, and protein complexes.

In some embodiments, the method includes pooling together a population of modified polynucleotide tags prior to said identifying, wherein said pooled population includes modified polynucleotide tags from different biological samples, from different polypeptide panels, or from different experimental conditions.

In some embodiments, the modifying process is repeated more than once prior to the identifying process.

In some embodiments, the at least one hybridization domain on the polynucleotide domain is complementary to at least one hybridization domain on the same polynucleotide.

In some embodiments, the population of polynucleotide tags is divided into separate aliquots prior to said amplifying of said polynucleotide duplex.

In some embodiments, the amplifying comprises a polymerase chain reaction or a quantitative polymerase chain reaction.

In an aspect, the method for detecting a polypeptide in a biological sample, comprises: (a) contacting a biological sample with at least one set of probes, wherein each probe comprises a polypeptide binding domain that specifically recognizes a polypeptide target and a polynucleotide domain that includes at least one unique barcode and at least one hybridization domain; (b) enabling each at least one set of probes to bind to each polypeptide target; (c) enabling the at least one hybridization domain to hybridize and form a polynucleotide duplex; (d) amplifying the polynucleotide duplex, comprising said unique barcodes, and generating a population of polynucleotide tags that identify the polypeptides; (g) modifying said polynucleotide tags by: (i) incubating said population of polynucleotide tags with extension primers capable of hybridizing and catalyzing extension of the polynucleotide tags and of incorporating at least one additional unique barcode into said polynucleotide tag wherein said at least one additional unique barcode identifies the sample source; (ii) amplifying said population of polynucleotide tags to incorporate the at least one additional unique barcode generating a population of modified polynucleotide tags that include at least two unique barcodes; (e) pooling separate populations of modified polypeptide tags into panels of pooled polypeptide tags; (f) modifying said panels of pooled polypeptide tags by: (i) incubating said panels of pooled polypeptide tags with extension primers capable of hybridizing and catalyzing extension of panels of pooled polypeptide tags and of incorporating at least one additional unique barcode; (ii) amplifying said panels of pooled polypeptide tags to incorporate the at least one additional unique barcode and generate a population of polypeptide tags that include at least three separate unique barcodes; and (g) identifying the polypeptide using said modified polynucleotide tags. In some embodiments, said at least one hybridization domain on said polynucleotide domain is complementary to at least one hybridization domain on the same polynucleotide. In some embodiments, said population of polynucleotide tags is divided into separate aliquots prior to said amplifying of said polynucleotide duplex. In some embodiments, said amplifying comprises a polymerase chain reaction or a quantitative polymerase chain reaction. In some embodiments, said modifying comprises a polymerase chain reaction or a quantitative polymerase chain reaction.

In an aspect, the present disclosure provides for a method for preparing polypeptides in a plurality of biological samples for sequencing, comprising: providing the plurality of biological samples from one or more subjects, wherein the biological samples comprise one or more polypeptides; for each biological sample of the plurality of biological samples, separating the sample into a plurality of sub-samples; contacting each sub-sample with a panel of probes to generate polynucleotide duplexes, wherein each probe comprises a polypeptide binding domain that specifically recognizes a polypeptide target and a polynucleotide domain that includes at least one target barcode and at least one hybridization domain; amplifying said polynucleotide duplexes, each comprising said target barcodes, to generate a population of target-barcoded polynucleotides; tagging each population of target-barcoded polynucleotides with a sample barcode to generate sample-barcoded polynucleotides; pooling the sample-barcoded polynucleotides of each sub-sample having the same panel of probes from different biological samples; for each pool of sample-barcoded polynucleotides, tagging the sample-barcoded polynucleotides with a panel barcode to generate pools of panel-barcoded polynucleotides; and sequencing a plurality of pooled panel-barcoded polynucleotides.

In an aspect, the present disclosure provides for a kit comprising a plurality of compositions and reagents to practice the methods disclosed herein. In some embodiments, the kits may comprise a plurality of probes for target molecule or polypeptide panels and primers for at least the sample and panel barcodes. In some embodiments, the kit comprises the sample and panel barcode oligonucleotides. Other amplification primers and/or sequencing adapters may also be included in the kit.

In certain embodiments, systems biology approaches directed to the study of complex diseases can integrate proteomics (proteins and their post-translational modifications) data with genomic, epigenomic, and transcriptomic data leading to new biological and insights of disease initiation, progression, malignant transformation, and therapeutic outcomes.

In some embodiments, the results of the systems and methods disclosed herein are used as an input to generate a report. The report may be in a paper or electronic format. For example, genetic results as determined by the methods and systems disclosed herein, such as the presence of a nucleic acid variant was detected in a sample, can be displayed directly in such a report. In some embodiments, only the presence or absence of a disease, such as cancer, is displayed in such a report.

The various steps of the methods disclosed herein, or steps carried out by the systems disclosed herein, may be carried out at the same or different times, in the same or different geographical locations, e.g., countries, and/or by the same or different people. In some embodiments, the report is communicated to a subject, for example, a subject who has cancer and has undergone testing by the methods and systems described herein, or to a healthcare professional, such as a physician treating the subject that has cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying Figures, which are incorporated in and constitute a part of this specification, illustrate several aspects described below and do not limit any claimed invention.

FIG. 1. The figure shows a standard proximity extension assay.

FIG. 2. The figure shows a panel/pool indexing PCR strategy of an embodiment of the present invention that increases throughput of immunoassays, such as proximity extension assays.

### DETAILED DESCRIPTION

### A. General

The present disclosure provides a method for significantly increasing the number of biological molecules that can be detected in fluids and processed using high throughput immunoassays.

While various embodiments of the disclosure have been shown and described herein, those skilled in the art will understand that such embodiments are provided by way of example only. Numerous variations, changes, and substitutions may occur to those skilled in the art without departing from the disclosure. It should be understood that various alternatives to the embodiments of the disclosure described herein may be employed.

### B. Definitions

The term “about” and its grammatical equivalents in relation to a reference numerical value can include a range of values up to plus or minus 10% from that value. For example, the amount “about 10” can include amounts from 9 to 11. The term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value “10” and any numerical value under 10, such as 9, 8, 5, 1, 0.5, and 0.1.

As used herein the singular forms “a”, “an”, and “the” can include plural referents unless the context clearly dictates otherwise. Thus, for example, reference to “a cell” can include a plurality of such cells and reference to “the culture” can include reference to one or more cultures and equivalents thereof known to those skilled in the art, and so forth. All technical and scientific terms used herein can have the same meaning as commonly understood to one of ordinary skill in the art to which this disclosure belongs unless clearly indicated otherwise.

The term “subject,” as used herein, generally refers to an animal, such as a mammalian species (e.g., human) or avian (e.g., bird) species, or other organism, such as a plant. More specifically, the subject can be a vertebrate, e.g., a mammal such as a mouse, a primate, a simian or a human. Animals include, but are not limited to, farm animals, sport animals, and pets. A subject can be a healthy individual, an individual that has or is suspected of having a disease or a pre-disposition to the disease, or an individual that is in need of therapy or suspected of needing therapy. A subject can be a patient.

The term “protein” or “polypeptide” (sometimes referred to as a “peptide” or “oligopeptide”) generally refers to a molecule that includes at least two amino acids joined by a peptide bond. A protein can be natural or synthetic. A protein may include one or more modified or non-natural amino acids or non-natural amino acid linkers. A protein may contain enantiomers of either form of an amino acid. Amino acids of a protein, and the protein in its entirety, may be modified naturally or synthetically and may include post-translational modifications. Different proteins may be distinguishable based on different: polynucleotides or the genes encoding them, isoforms, primary sequence composition, polynucleotide sequence length, or post-translational modifications, even if two proteins are expressed from the same gene.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence is referred to as the “complement” of the first sequence. The term “hybridizable” as applied to a polynucleotide refers to the ability of the polynucleotide to form a complex that is stabilized via hydrogen bonding between the bases of the nucleotide residues in a hybridization reaction.

“Barcode” or “tag” refers to nucleic acids that contain an identification motif, for example a polypeptide target-level identification motif (“target barcode”), a sample-level identification motif (“sample barcode”) or a panel-level identification motif (“panel barcode”). A barcode can include a nucleic acid sequence of any length, such as at least 2, 3, 4, 5, 6, 7, 8, 9, 10, 20, 30, 40, 50, 60, 70, 80, 90, 100 or more nucleotide bases.

“Probe”, as used herein, refers to a molecule that can bind specifically to a particular domain or a motif on a polypeptide and that includes a functionality. The functionality can be a polynucleotide label, a fluorescent label, a binding moiety (biotin), or a solid support (a magnetically attractable particle or a chip).

### C. Workflow

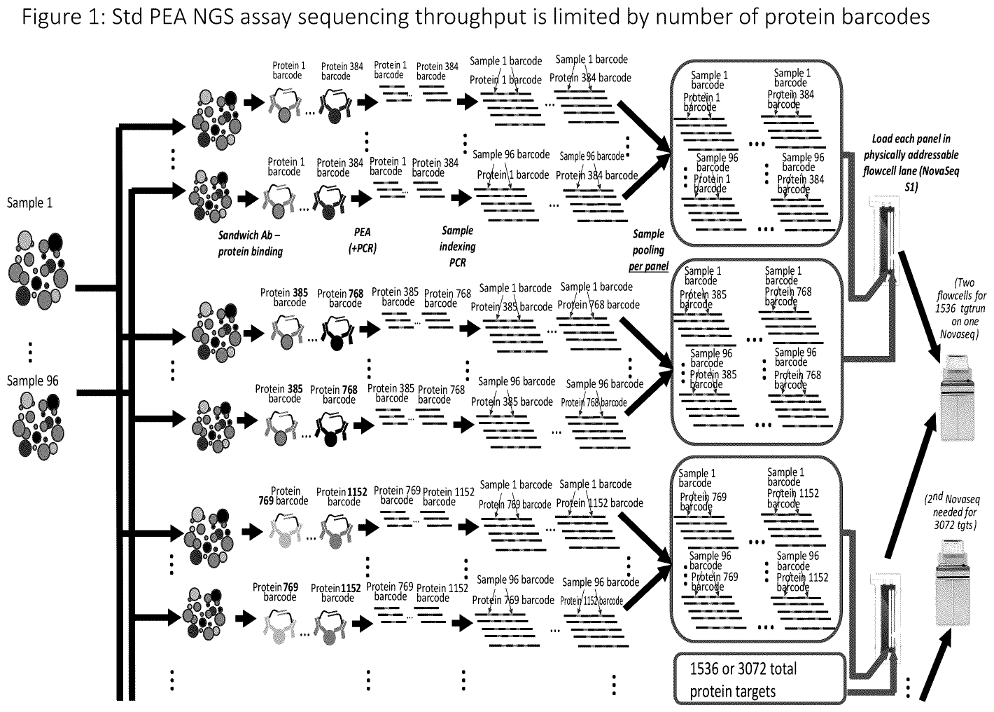

According to certain aspects of the disclosure, identification of and assays for specific molecules or polypeptides, such as proteins, that are present in biological samples are achieved by using an improved immunoassay combined with high throughput sequencing. One such immunoassay is a Proximity Extension Assay (PEA). A standard PEA is shown in FIG. 1, which includes the following steps: binding of matched PEA antibody probes in a manner that enables hybridization of nucleotides to create a barcode attached to the probes; extension and PCR amplification of the nucleotide barcodes that identify a given protein; incubation of these barcodes with primers that include unique sequences that identify sample sources; PCR amplification of the nucleotide barcodes that contain protein and sample identifiers; pooling of the resulting nucleotide barcodes from sub-samples contacted with the same panel of probes; followed by sequencing, wherein each panel is loaded in a different flow cell or in physically addressable flow cell lanes.

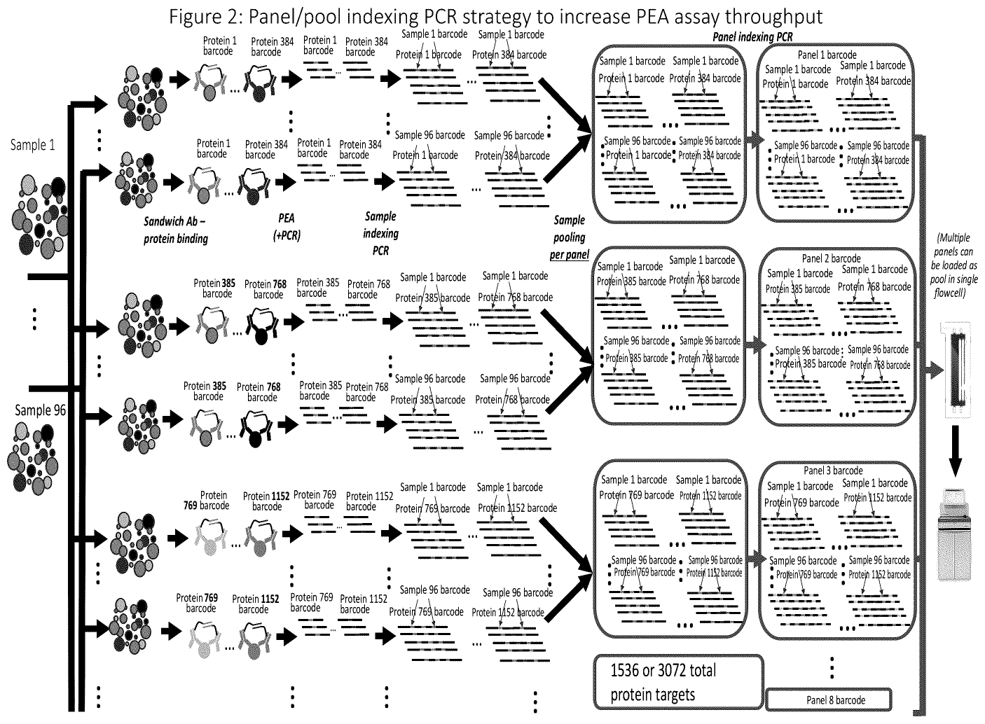

One of the issues with typical immunoassays like the PEA is the inefficiency of sequencing costs caused by the limit in the number of DNA-encoded protein barcodes. Accordingly, the present disclosure provides an enhanced PEA-type assay that increases sequencing efficiency by introduction of panel barcodes. An embodiment of such a workflow is shown in FIG. 2.

Methods disclosed herein can comprise providing molecules or polypeptides of interest from a plurality of biological samples from one or more subjects. In some embodiments, the biological sample is obtained from a tissue sample. In other embodiments, the biological sample is from a bodily fluid, such as blood, plasma, and serum.

In various embodiments, the polypeptides of interest are derived from tumor cells, cells in another disease state, or cells that are altered due to the presence of a disease in the subject from which the cells are obtained. In some embodiments, one or more target proteins are derived from cell types not normally present in the type of bodily sample obtained from a subject. In some embodiments, the molecules or polypeptides of interest are blood-based (i.e., a blood protein biomarker).

The number of polypeptides to be identified can vary depending on the context of the analysis to be performed. In some embodiments, polypeptides of interest may comprise at least 2 polypeptides, at least 3 polypeptides, at least 4 polypeptides, at least 5 polypeptides, at least 10 polypeptides, at least 20 polypeptides, at least 30 polypeptides, at least 40 polypeptides, at least 50 polypeptides, at least 100 polypeptides, at least 200 polypeptides, at least 300 polypeptides, at least 400 polypeptides, at least 500 polypeptides, at least 1,000 polypeptides, at least 2,000 polypeptides, at least 3,000 polypeptides, at least 4,000 polypeptides, at least 5,000 polypeptides, at least 10,000 polypeptides, and at least 15,000. In some embodiments, the number of polypeptides of interest is between 1,000 and 5,000. In some embodiments, the number of polypeptides of interest is between 1,000 and 10,000. In some embodiments, the number of polypeptides of interest is between 1,000 and 15,000. In some embodiments, the number of polypeptides is more than 15,000.

In some embodiments, the methods of the present disclosure are directed to detecting one or more molecules or polypeptides in a plurality of biological samples. In some embodiments, the plurality of biological samples comprises at least 2 samples, at least 5 samples, at least 10 samples, at least 15 samples, at least 20 samples, at least 30 samples, at least 40 samples, at least 50 samples, at least 60 samples, at least 70 samples, at least 80 samples, at least 90 samples, and at least 100 samples. In some embodiments, the plurality of samples comprises more than 100 samples. In some embodiments, the plurality of samples may be obtained from the same subject or from different subjects.

According to various embodiments, the polypeptide targets of interest for detection and/or quantitation are cancer-associated biomarkers. Several cancer-associated biomarkers are known and can be detected and quantitated according to embodiments of the present disclosure. In various embodiments, the cancer-associated biomarker is a peptide or protein selected from, for example, oncofetal antigens (e.g., CEA, AFP), glycoprotein antigens or carbohydrate antigen (e.g. CA125, CA 19.9, CA 15-3), enzymes (e.g., PSA, ALP, NSE), hormone receptors (e.g., ER, PR), hormones (e.g., b-hCG, calcitonin), or other known biomolecules (e.g., VMA, 5HIAA, Mucins 1-24)). In various examples, the cancer-associated biomarker is associated one or more of colorectal cancer, liver cancer, ovarian cancer, lung cancer, pancreatic cancer, stomach cancer, bladder cancer, or breast cancer.

In some embodiments, the polypeptide of interest is a glycoprotein carbohydrate. In some embodiments, the polypeptide of interest is selected from RBI, TP53, PTEN, NF1, BRCA1, CEACAM1, CEACAM5, CEACAM6, EGFR, ErbB2, ErbB3, ErbB4, B-catenin, PD-L1, CTLA4, NYESO1, mesothelin, CA15-3, CA19-9, CA-125, CA27-29, and CA-72-4. In some embodiments, the polypeptide of interest is a protein known to show altered post-translational modification (e.g., altered glycosylation) in cancer. In some embodiments, the polypeptide of interest is a cell type marker, such as an immune cell type marker or solid tissue cell type marker. In some embodiments, the solid tissue cell type marker is a marker present in colon, lung, breast, skin, prostate, stomach, pancreas, or liver cells.

In various embodiments, the polypeptide of interest is a cancer associated peptide or protein marker are selected from 1p/19q deletion, HIAA, ACTH, AE1,3, ALK(D5F3), AFP, APC, ATRX, BOB-1, BCL-6, BCR-ABL1, beta-hCG, BF-1, BTAA, BRAF, GCDFP-15, BRCA1, BRCA2, b72.3, c-MET, calcitonin, CALR, calretinin, CA125, CA27.29, CA 19-9, CEA M, CEA P, CEA, CBFB-MYH11, CALA, c-Kit, syndical-1, CD14, CD15, CD19, CD2, CD20, CD200, CD23, CD3, CD30, CD33, CD4, CD45, CD5, CD56, CD57, CD68, CD7, CD79A, CD8, CDK4, CDK2, chromogranin A, creatine kinase isoenzymes, Cox-2, CXCL 13, cyclin D, CK 19, CYFRA 21-1, CK 20, CK5,6, CK 7, CAM 5.2, DCC, des-gamma-carboxy prothrombin, E-cadherin, EGFR T790M, EML4-ALK, ERBB2, ER, ESR1, FAP, gastrin, glucagon, HER-2/neu, SDHB, SDHC, SDHD, HMB45, HNPCC, HVA, beta-hCG, HE4, FBXW7, IDH1 R132H, IGH-CCND1, IGHV, IMP3, LOH, MUM1/IRF4, JAK exon 12, JAK2 V617F, Ki-67, KRAS, MCC, MDM2, MGMT, melan A, MET, metanephrines, MSI, MPL codon 515, Muc-1, Muckiest-4, MEN2, MYC, MYCN, MPO, myf4, myoglobin, myosin, napsin A, neurofilament, NSE P, NMP22, NPM1, NRAS, Oct 2, p16, p21, p53, pancreatic polypeptide, PTH, Pax-5, PAX8, PCA3, PD-L1 28-8, PIK3CA, PTEN, ERCC-1, Ezrin, STK11, PLAP, PML/RARa translocation, PR, proinsulin, prolactin, PSA, PAP, PGP, RAS, ROS1, 5-100, S100A2, S100B, SDHB, serotonin, SAMD4, MESOMARK, squamous cell carcinoma antigen, SS18 SYT 18i1i, synaptophysin, TIA-1, TdT, thyroglobulin, TNIK, TP53, TTF-1, TNF-alpha, TRAFF2, urovysion, VEGF, or combinations thereof.

In some embodiments, the polypeptide of interest may be selected form an interleukin protein family, including IL-1, IL-2, IL-6, IL10, IL-12, IL-17 families.

In some embodiments, the polypeptide of interest may include a receptor for advanced glycation end products (RAGE).

In some embodiments, the polypeptide of interest is associated with a disease, two or more of a plurality of the polypeptide of interest are associated with a disease, or each of a plurality of the polypeptide of interest is associated with a disease, e.g., cancer. In some embodiments, the polypeptide of interest is a protein that is differentially post-translationally modified in tumor cells relative to healthy cells of the same tissue type. In some embodiments, the polypeptide of interest is upregulated in tumor cells relative to healthy cells of the same tissue type.

In some embodiments, the subject or subjects from which the biological sample or samples are collected are known to have a disease, such as cancer. In other embodiments, the subject or subjects are not known to have a disease.

Referring to FIG. 2, in certain embodiments, a sample comprising the molecules or polypeptides of interest is separated into a plurality of sub-samples. The sample, such as Sample 1, is split (e.g., by aliquoting) into sub-samples. The separation of the sample into sub-samples is based at least on the number of molecules or polypeptides to be analyzed. Probes for detecting the molecules or polypeptides of interest make up a target panel. Each sub-sample is thus representative of a panel. Each probe of a panel comprises a polypeptide binding domain that specifically recognizes a polypeptide target, and a panel of probes thus recognizes a set of polypeptide targets. For example, Sample 1 is separated into a sub-sample that has a panel comprising target proteins 1-384, a second sub-sample for proteins 385-768, a sub-sample for proteins 768-1152, and so forth. Other samples are also separated into sub-samples (for example, as shown, Sample 96). In some embodiments, the sample is separated into at least 2 sub-samples, at least 3 sub-samples, at least 4 sub-samples, at least 5 sub-samples, at least 6 sub-samples, at least 7 sub-samples, at least 8 sub-samples, at least 9 sub-samples, at least 10 sub-samples, at least 15 sub-samples, or at least 20 sub-samples. In some embodiments, one sample may be separated into a different number of sub-samples than another sample.

A plurality of the sub-samples is then contacted with the probe panels. In certain embodiments, the probes are proximity probes that comprise antibodies, which are incubated under conditions that allow specific binding of the antibodies to their target antigens. Antigen specific Fab fragments, Fab′ fragments, F(ab)′2 fragments, single chain Fv proteins (“scFv”), or disulfide stabilized Fv proteins (“dsFv”) are utilized to identify and bind to specific antigens on a given protein to determine whether the protein is present in a sample. Matched pairs of oligonucleotide-labeled antibodies are used to bind to their target antigens on a protein in a pairwise manner, and each antibody has a distinct oligonucleotide fragment covalently attached to it.

In certain embodiments, the probes comprise nucleotide sequences. The nucleotide fragments (which are attached to the antibodies and contain distinct complementary sequences) hybridize and form a nucleotide pair. Each antigen thereby generates a nucleic acid (e.g., DNA) sequence, and, therefore, the resulting amplicons serve as “target barcodes” that identify which proteins are present in a biological sample. As used herein, a “target barcode” (or sometimes otherwise referred to as “protein barcode”) is specific for a given protein target within a panel of probes. According to certain aspects of the invention, target barcodes can be shared across different panels of probes. For example, the same set of target barcodes can be used in the first panel of probes as in the second (or further) panel of probes, for example the same set of 384 target barcodes can be used across two or more panels of probes. As shown in FIG. 2, “Protein 1 barcode” for Sample 1 in top workflow is the same nucleic acid sequence as that of “Protein 385 barcode” for Sample 1 in the second line workflow from the top.

The number of target barcodes is limited at least in part by costs associated with the number of proteins that are to be analyzed. For example, assays directed to detecting more than 1,000 proteins would require more than 1,000 unique barcode sequences. Constructing such a large number of barcode sequences is prohibitively expense and cumbersome. Therefore, to help minimize costs, target barcodes are used across different panels. For example, in FIG. 2 there is a set of different barcodes (384), and each barcode in the set is associated with a probe recognizing a different protein. These probes are in a panel recognizing a number of proteins that matches the number of barcodes (384). There is then a plurality of panels, each panel comprising the same set of barcodes but different probes. In some embodiments, the panels may share barcode sequences and protein targets. For example, a panel targeting breast cancer may comprise one or more protein targets that overlap with a panel directed to colorectal cancer if a specific protein is indicative of both cancers.

In certain embodiments, the barcode sequences may be used alone or in combination with other sequence information from the amplicons for detecting the molecule or polypeptide of interest. For example, the nucleotide sequences of the probe may be optimized to hybridize to its complementary sequence on another probe, but also reduced in sequence length where the target barcode sequence can be used with non-complementary portions of the nucleotide fragment to identify and detect a protein.

PCR amplification is used to amplify the nucleotide pairs and to create a population of nucleic acid amplicons. In certain embodiments, the amplicons comprise the polynucleotide target barcode. In certain embodiments, quantitative PCR (qPCR) or real time PCR can be used to evaluate the concentrations of a particular protein in a sample using the concentration of a given amplicon.

Once polynucleotide target barcodes have been generated, the amplicons containing the polynucleotide target barcodes from a given sub-sample are modified by incorporation of a “sample barcode,” wherein sub-samples derived from the same sample receive the same sample barcode and which differs from sample barcodes of other samples. As used herein, a “sample barcode” (sometimes also referred to as “sample indexing”) helps differentiate molecules derived from a given sample from molecules derived from another sample or samples. According to certain aspects of the invention, amplicons comprising the polynucleotide target barcodes deriving from the same sample are modified with the same sample barcode, whereas polynucleotide sample barcode deriving from a different sample are modified with a different sample barcode. The sample barcode may be introduced into the amplicons by any known method, such as the use of a set of PCR primers. The amplicons comprising both the target barcode and sample barcode are then amplified.

After sample indexing, sub-samples of different biological samples and which have been contacted with the same panel of probes may be pooled. The amplicons comprising the target barcode and sample barcode in a given pool are then further modified by incorporation of a panel barcode, wherein panel barcodes differ between pools. As used herein, a “panel barcode” (or sometimes referred to as a “panel index”) helps differentiate molecules contacted with a given panel of probes from molecules contacted with another panel of probes. According to the certain aspects of the invention, amplicons generated using the same panel of probes are modified with the same panel barcode, whereas amplicons generated using a different panel of probes are modified with a different panel barcode. For example, in the last step of the workflows prior to sequencing in FIG. 2, each panel barcode comprises a different nucleic acid sequence to specifically identify that particular panel (e.g., the “Panel 1 barcode” comprises a different nucleic acid sequence than the “Panel 2 barcode,” and so forth). The panel barcodes may be incorporated into the sequence of the pooled amplicons by any known method, such as by PCR amplification with primers. The panel indexing PCR step thus generates amplicons comprising a target barcode, sample barcode, and panel barcode.

Alternatively, in some embodiments, the panel-indexing may occur without pooling and/or before or at the same time as sample-indexing. In some embodiments, the primers may comprise both sample and panel barcode sequences.

A plurality of the amplicons from the panel indexing step are then prepared and loaded into a nucleic acid sequencer, such as a next generation sequencer, and undergo sequencing. Advantageously, and in contrast to standard PEA approaches as illustrated in FIG. 1, panel indexing according to embodiments of the present disclosure allows pooling across panels before sequencing, such that, for example, multiple panels can be loaded as a pool in a single flow cell. This saves on sequencing costs relative to standard PEA approaches, as shown in FIG. 1, where each panel must be loaded in separate flow cells or in physically addressable flow cell lanes. The cost savings become even more significant the larger the number of proteins targets become, i.e., the more panels that are used.

Sequencing may be carried out on any suitable sequencing platform. Nucleic acid sequencers from Illumina (e.g., a Novaseq® sequencer), Pacific Biosciences, Oxford Nanopore, Complete Genomics, Thermo Fisher, Ion Torrent, or the like may be used. The amplicons of the disclosed methods of the invention are prepared according to the type of sequencer, such as attaching sequencing adapters to the amplicons (e.g., by ligation or PCR amplification). In certain embodiments, the adapters are Y-shaped adapters or hairpin loop adapters. The adapters may comprise sequences that will allow for binding to complementary sequences on a flow cell or surface (e.g., a surface of a bead), or otherwise permit the sequencing process to be initiated. In certain embodiments, the primers used may be standard index primers or customized primers. Advantageously, in some embodiments, the sample barcodes/index used in the disclosed methods of the invention may be shorter in length compared to primers currently used in standard PEA approaches, which typically contain NGS flow cell primers (P5/P7), sequencing read primers, sample index and a hybridization sequence. Having a further step for panel indexing allows for the sample index to be reduced in length, for example, from 80 nucleotides to 50 nucleotides, thus making the sample indexing PCR more efficient.

### D. Analysis

Sequencing data comprising the nucleic acid sequences of the panel-indexed amplicons is collected. Analysis of the sequencing data is then used to detect and resolve the identity of the target polypeptides and the sample source using at least a combination of the target barcode, sample barcode and/or panel barcode.

In certain embodiments, identity of the target polypeptides and other proteomic information from the target proteins may be used in various applications, including screening for a disease or treating the disease.

In certain embodiments, the proteomic information can also be integrated with histology, imaging, genetic, and/or epigenetic information such as methylation patterns, transcription factor binding sites (TFBS), and histone modifications collected from other analyses. For example, a neural network approach can integrate multiple layers of proteomic and epigenomic information to predict predictive features, using deep learning or neural networks to handle high-dimensional data.

### E. Applications

The present methods can be used to diagnose presence of conditions, particularly cancer, in a subject, to characterize conditions (e.g., selection of appropriate treatment or staging cancer or determining heterogeneity of a cancer), monitor response to treatment of a condition, effect prognosis risk of developing a condition or subsequent course of a condition.

Various cancers may be detected using the present methods. In some embodiments, the cancer is detected from biological samples comprising circulating proteins. In cancer patients, serum-antibody profiles change, as well as autoantibodies against the cancerous tissue are generated. Those profile-changes provide much potential for tumor-associated antigens as markers for early diagnosis of cancer. The immunogenicity of tumor associated antigens is conferred to mutated amino acid sequences, which expose an altered non-self-epitope. Other explanations are also implicated of this immunogenicity, including alternative splicing, expression of embryonic proteins in adulthood (e.g. ectopic expression), deregulation of apoptotic or necrotic processes (e.g. overexpression), abnormal cellular localizations (e.g. nuclear proteins being secreted). Examples of epitopes of the tumor-restricted antigens, encoded by intron sequences (e.g. partially unspliced RNA were translated) have been shown to make the tumor associated antigen highly immunogenic. Example markers are suitable protein antigens that are overexpressed in tumors. The markers usually cause an antibody reaction in a patient. Accordingly, in some embodiments, the panels may comprise a plurality of cancer-protein biomarkers. In some embodiments, a panel may be directed to a specific disease or cancer type, e.g., colorectal cancer.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

Proteomic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive or dormant. The system and methods of this disclosure can be useful in determining disease progression.

Accordingly, the present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may result in changes in levels of different cancer-related protein levels and/or increase the amount of copy number variation, rare mutations, and/or cancer-related epigenetic signatures (such as hypermethylated regions or hypomethylated regions) detected in, e.g., a sample from a subject, such as detected in a subject's blood (such as circulating proteins or in DNA isolated from a buffy coat sample or any other sample comprising cells, such as in a blood sample (e.g., a whole blood sample, a leukapheresis sample, or a PBMC sample) from the subject) if the treatment is successful as more cancer cells may die and shed proteins or DNA, or, e.g., if a successful treatment results in an increase or decrease in the quantity of a specific cancer-related proteins in the blood and an unsuccessful treatment results in no change. In other examples, this may not occur. These changes may be useful in selecting a therapy.

Additionally, if a cancer is observed to be in remission after treatment, the present methods can be used to monitor the likelihood of residual disease or the likelihood of recurrence of disease.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, a sample is obtained from a subject who was previously diagnosed with the cancer and received one or more previous cancer treatments, optionally wherein the sample is obtained at one or more preselected time points following the one or more previous cancer treatments. In some embodiments, a sample is obtained from a subject who was previously diagnosed with the cancer, and the sample is obtained from the subject before the subject receives a cancer treatment. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Typically, the disease under consideration is a type of cancer, such as any referred to herein. The types and number of cancers that may be detected may include blood cancers, brain cancers, eye cancers, oral cancers, head and neck cancers, gallbladder cancers, endometrial cancers, ovarian cancers, uterine cancers, prostate cancers, esophageal cancers, lung cancers, skin cancers, nose cancers, throat cancers, liver cancers, bone cancers, lymphomas, leukemias, pancreatic cancers, skin cancers, gastrointestinal cancers, bowel cancers, colorectal cancers, colon cancers, rectal cancers, thyroid cancers, bladder cancers, kidney cancers, mouth cancers, stomach cancers, breast cancers, solid state tumors, heterogeneous tumors, homogenous tumors and the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. In some embodiments, the cancer is a hematological cancer. In other embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine. In some embodiments, the cancer is a type of cancer that is not a hematological cancer, e.g., a solid tumor cancer such as a carcinoma or sarcoma.

The present methods can be used to generate a profile, fingerprint, or set of data that is a summation of information derived from different cells in a heterogeneous disease. This set of data may comprise proteomic data obtained from the methods disclosed herein. In some embodiments, other analytes from the sample from the subject, such as DNA or RNA (e.g., cell-free DNA or cell-free RNA), and other information from those analytes, such as epigenomic information (e.g., methylation or fragmentomic information) may also be used to in generating the profile.

Proteomic data can also be used for characterizing a specific form of cancer. Cancers are often heterogeneous in both composition and staging. Proteomic profile data may allow characterization of specific sub-types of cancer may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The methods of this disclosure may be useful in determining disease progression.

Further, the methods of this disclosure may be used to characterize the heterogeneity of an abnormal condition in a subject. Such methods can include, e.g., generating a protein profile using proteins derived from the subject. In some embodiments, the proteins are circulating proteins from a blood, plasma, or serum sample obtained from the subject. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose RNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardio facial syndrome, WAGR syndrome, Wilson disease, or the like.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

In some embodiments, the methods disclosed herein comprise determining the likelihood that the subject from which the sample was obtained has cancer, precancer, an infection, transplant rejection, or other diseases or disorder that is related to changes in proportions of types of immune cells. As discussed herein, comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed above may further comprise any compatible feature or features set forth elsewhere herein, including in the section regarding methods of determining a risk of cancer recurrence in a subject and/or classifying a subject as being a candidate for a subsequent cancer treatment.

The present methods can also be used to monitor therapy. For example, a successful treatment can initially be associated with an increase in markers associated with the disease or cancer for which the therapy has been administered to treat in circulating proteins as cancer cells die and release proteins to the circulation. This initial increase can be followed by a decrease reflecting fewer if any remaining cancer cells to release proteins. There can also be a subsequent increase in circulating proteins following a period of remission providing an indication of recurrence of the cancer.

The present methods can also be used for detecting genetic variations in conditions other than cancer. Immune cells, such as B cells, undergo copy number variation associated with certain diseases. Clonal expansions can be monitored using copy number variation detection as a measure of disease progression. The present methods may be used to determine or profile rejection activities of the host body, as immune cells attempt to destroy transplanted tissue to monitor the status of transplanted tissue as well as altering the course of treatment or prevention of rejection. Copy number variation or variant nucleotide can be used to determine how a population of pathogens are changing during the course of infection. For example, during chronic infections, such as HIV/AIDs or Hepatitis infections, viruses may change life cycle state and/or mutate into more virulent forms during the course of infection.

The present methods can be used to generate or profile, fingerprint or set of data that is a summation of genetic information derived from different cells in a heterogeneous disease. This set of data may comprise copy number variation and nucleotide variation or both.

The present methods can be used to diagnose, prognose, monitor or observe cancers or other diseases of fetal origin. That is, these methodologies can be employed in a pregnant subject to diagnose, prognose, monitor or observe cancers or other diseases in an unborn subject whose DNA and other nucleic acids may co-circulate with maternal molecules.

In certain embodiments, the present methods can be used to determine minimal residual disease (MRD) of a subject. In some embodiments, the methods may be directed to determining MRD by using a tissue-informed assay (i.e., using a tissue sample collected from a patient to determine a personalized panel to enrich for one or more target proteins, and/or with genomic and/or epigenomic variants in a subsequent blood sample from the patient) or a tissue-naïve assay.

In certain embodiments, the present methods can integrate proteomic data (proteins and their post-translational modifications) with genomic, epigenomic, transcriptomic, fragmentomic, immunological, histological, and/or other analyte-specific data to determine disease initiation, progression, malignant transformation, and therapeutic outcomes.

Advantageously, in certain embodiments, since the methods described herein free-up flow cells for NGS sequencing, additional analytes, such as cell-free nucleic acids may be use for parallel genomic and/or epigenomic analysis. In some embodiments, the protein-derived amplicons from the pooled panel may be further pooled with amplicons from nucleic acids from the sample, such as cell-free DNA.

1. Methods of Determining a Risk of Cancer Recurrence in a Subject and/or Classifying a Subject as being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting proteins and/or nucleic acids (e.g., originating or derived from an immune cell or a cancer cell) from the subject diagnosed with the cancer at one or more preselected timepoints following one or more previous cancer treatments to the subject. Similarly, any of such methods may comprise collecting proteins and/or nucleic acids (e.g., originating or derived from an immune cell or a cancer cell) from the subject diagnosed with the cancer at one or more preselected timepoints preceding one or more previous cancer treatments to the subject. The subject may be any of the subjects described herein. The nucleic acids may be DNA or RNA from a sample comprising cells or a blood sample (e.g., a buffy coat sample, a whole blood sample, a leukapheresis sample, or a PBMC sample). The nucleic acid molecules may comprise DNA or RNA obtained from a tissue sample.

Any of such methods may comprise capturing target regions from DNA or RNA (or cDNA prepared from the RNA) from the subject whereby a captured set of nucleic acid molecules is produced.

In any of such methods, the previous cancer treatment may comprise surgery, administration of a therapeutic composition, and/or chemotherapy.

Any of such methods may comprise sequencing the captured nucleic acid molecules, whereby a set of sequence information is produced.

Methods of determining a risk of cancer recurrence in a subject may comprise determining a cancer recurrence score that is indicative of the presence or absence, or amount, of nucleic acid molecules originating or derived from an immune cell or a cancer cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, determining the cancer recurrence score may comprise determining at least a first subscore indicative of the levels of particular immune cell types present based on expression levels of target proteins. In some embodiments, determining the cancer recurrence score may comprise determining at least a first subscore indicative of the levels of particular immune cell types present based on whole transcriptome sequencing.

In some embodiments, any of such methods may comprise determining a fraction of tumor from the fraction of molecules in the set of sequence information that indicate one or more features indicative of origination from a tumor cell or an immune cell. This may be done for molecules corresponding to some or all of the target proteins, e.g., including, e.g., molecules comprising alterations consistent with cancer. A determination that a fraction of tumor or immune cell proteins is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 2. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening), such as based on changes in levels of different immune cell types, including rare immune cell types, in samples collected from a subject over time. In some embodiments, monitoring comprises analysis of at least two samples collected from a subject at least two different time points as described herein.

The methods according to the present disclosure can also be useful in predicting a subject's response to a particular treatment option. Successful treatment options may result in an increase or decrease in the quantity of a specific immune cell type in the blood, or in the expression of one or more of the plurality of genes of a target gene set, and an unsuccessful treatment may result in no change. In other examples, this may not occur. In another example, certain treatment options may be correlated with genetic profiles of cancers over time. This correlation may be useful in selecting a therapy for a subject.

As disclosed herein, methods are provided for monitoring one or more aspects of a condition in a subject over time, such as but not limited to, a subject's response to receiving a treatment for a condition (such as a response to a chemotherapeutic or immunotherapeutic). Thus, some embodiments of the disclosed methods further comprise evaluating or monitoring a response to a treatment in the subject. In some embodiments, the evaluating or monitoring the response to the treatment in the subject comprises comparing the expression levels for the target gene set comprising a plurality of target genes that are differentially expressed in a sample from the subject collected at least a first time point and a sample from the subject collected at least a second time point. In some embodiments, the evaluating or monitoring the response to the treatment in the subject comprises comparing the quantities of the immune cell types in a sample from the subject collected at least a first time point and a sample from the subject collected at least a second time point. In some embodiments, the first time point is a time point prior to administration of the treatment to the subject, and the second time point is a time point after the administration of the treatment to the subject. In some embodiments, the first time point is a time point after administration of the treatment to the subject, and the second time point is a time point after the administration of the treatment to the subject and after the first time point.

In certain embodiments, one or more samples is collected from the subject at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples comprising cells or a blood sample (such as one or more whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples comprising cells or one or more blood samples, e.g., one or more buffy coat samples, whole blood samples, leukapheresis samples, or PBMC samples, are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### F. Therapies and Related Administration

In certain embodiments, the methods disclosed herein relate to identifying and administering therapies, such as customized therapies, to patients or subjects. In some embodiments, determination of the presence or absence or levels of polypeptides and/or genetic or epigenomic variations, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods.

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non- Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha- 1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

In certain embodiments, the therapies can include one or more of treatments for target therapies, including abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab- vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aligopa), crizotinib (Xalkori), dabrafenib (Tafmlar), dabrafenib mesylate (Tafmlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxetan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane 1 131 (Azedra), ipilimumab (Yervoy), isatuximab-irfc (Sarclisa), ivosidenib (Tibsovo), ixazomib citrate (Ninlaro), lanreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligeo), mosunetuzumab-axgb (Lunsumio), moxetumomab pasudotox-tdfk(Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride(Votrient), pembrolizumab (Keytruda), pemigatinib(Pemazyre), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate(Rubraca), ruxolitinib phosphate (Jakafi), sacituzumab govitecan-hziy (Trodelvy), selinexor (Xpovio), selpercatinib (Retevmo), selumetinib sulfate (Koselugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-erzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), trametinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti- tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intrarticular, which administration may include tablets, capsules, granules, aqueous suspensions, gels, sprays, suppositories, salves, ointments, or the like.

In certain embodiments, the present methods are also useful in determining the efficacy of particular treatment options. For example, the number of variations detected, irrespective of their precise identity, is a predictor of amenability to immunotherapy because the mutations create neoepitopes that can be subject of immune attack (see e.g., US20200370129).

In certain embodiments, the panels comprise molecules or polypeptides of interest that are associated with a specific target for a drug to be administered to treat the subject. In some embodiments, the drug is used to treat a subject having a specific cancer type. In some embodiments, the cancer type, drug, and drug target may be selected from Table 1, below.

TABLE 1

| Cancer type | Drug | Target |
|---|---|---|
| Breast | abemaciclib (Verzenio) | CDK4, CDK6 |
| Prostate | abiraterone acetate | CYP17A1 |
| | (Zytiga) | |
| Leukemia | acalabrutinib | BTK |
| | (Calquence) | |
| Lymphoma | acalabrutinib | BTK |
| | (Calquence) | |
| Lung | adagrasib (Krazati) | KRAS G12C |
| Breast | ado-trastuzumab | Tubulin, Her2 |
| | emtansine (Kadcyla) | |
| Lung | afatinib dimaleate | EGFR + EGFR exon 21 L858R mutation + |
| | (Gilotrif) | EGFR-Ex19del + HER2 + HER4 |
| Lung | alectinib (Alecensa) | ALK, RET |
| Leukemia | alemtuzumab | CD52 |
| | (Campath) | |
| Skin | alitretinoin (Panretin) | RARα + RARβ2 + RARγ |
| Soft tissue sarcoma | alitretinoin (Panretin) | RARα + RARβ2 + RARγ |
| Breast | alpelisib (Piqray) | PI3Kα |
| Lung | amivantamab-vmjw | EGFR + c-MET |
| | (Rybrevant) | |
| Breast | anastrozole (Arimidex) | Aromatase |
| Prostate | apalutamide (Erleada) | AR |
| Leukemia | asciminib hydrochloride | Bcr-Abl |
| | (Scemblix) | |
| Bladder | atezolizumab | PDL1 |
| | (Tecentriq) | |
| Liver and bile duct | atezolizumab | PDL1 |
| | (Tecentriq) | |
| Lung | atezolizumab | PDL1 |
| | (Tecentriq) | |
| Skin | atezolizumab | PDL1 |
| | (Tecentriq) | |
| Soft tissue sarcoma | atezolizumab | PDL1 |
| | (Tecentriq) | |
| Gastrointestinal | avapritinib (Ayvakit) | PDGFRα + c-Kit |
| Systemic mastocytosis | avapritinib (Ayvakit) | PDGFRα + c-Kit |
| Bladder | avelumab (Bavencio) | PDL1 |
| Endocrine and | avelumab (Bavencio) | PDL1 |
| neuroendocrine tumors | | |
| Kidney | avelumab (Bavencio) | PDL1 |
| Skin | avelumab (Bavencio) | PDL1 |
| Lymphoma | axicabtagene ciloleucel | CD19 |
| | (Yescarta) | |
| Kidney | axitinib (Inlyta) | VEGFR1 + VEGFR2 + VEGFR3 |
| Lymphoma | belinostat (Beleodaq) | HDAC |
| Brain | belzutifan (Welireg) | HIF-2A |
| Kidney | belzutifan (Welireg) | HIF-2A |
| Pancreatic | belzutifan (Welireg) | HIF-2A |
| Ovarian epithelial, | bevacizumab (Avastin) | VEGF-A |
| fallopian tube, and | | |
| primary peritoneal | | |
| Brain | bevacizumab (Avastin) | VEGF-A |
| Cervical | bevacizumab (Avastin) | VEGF-A |
| Colorectal | bevacizumab (Avastin) | VEGF-A |
| Kidney | bevacizumab (Avastin) | VEGF-A |
| Liver and bile duct | bevacizumab (Avastin) | VEGF-A |
| Lung | bevacizumab (Avastin) | VEGF-A |
| Lymphoma | bexarotene (Targretin) | RXRs |
| Skin | binimetinib (Mektovi) | MEK1, MEK2 |
| Leukemia | blinatumomab | CD19, CD3 |
| | (Blincyto) | |
| Lymphoma | bortezomib (Velcade) | Proteasome |
| Multiple myeloma | bortezomib (Velcade) | Proteasome |
| Leukemia | bosutinib (Bosulif) | BCR-ABL, SRC |
| Lymphoma | brentuximab vedotin | CD130, Tubulin |
| | (Adcetris) | |
| Leukemia | brexucabtagene | CD19 |
| | autoleucel (Tecartus) | |
| Lymphoma | brexucabtagene | CD19 |
| | autoleucel (Tecartus) | |
| Lung | brigatinib (Alunbrig) | ALK, EGFR, FLT3, IGF-1R, ROS1 |
| Prostate | cabazitaxel (Jevtana) | Tubulin |
| Kidney | cabozantinib-s-malate | AXL + RET + ROS1 + TYRO3 + Tie-2 + |
| | (Cabometyx) | TrkB + VEGFR1 + VEGFR2 + VEGFR3 + |
| | | c-Kit + c-Met |
| Liver and bile duct | cabozantinib-s-malate | AXL + RET + ROS1 + TYRO3 + Tie-2 + |
| | (Cabometyx) | TrkB + VEGFR1 + VEGFR2 + VEGFR3 + |
| | | c-Kit + c-Met |
| Thyroid | cabozantinib-s-malate | AXL + RET + ROS1 + TYRO3 + Tie-2 + |
| | (Cometriq) | TrkB + VEGFR1 + VEGFR2 + VEGFR3 + |
| | | c-Kit + c-Met |
| Lung | capmatinib | c-Met |
| | hydrochloride | |
| | (Tabrecta) | |
| Multiple myeloma | carfilzomib (Kyprolis) | protoasome |
| Lung | cemiplimab-rwlc | PD-1 |
| | (Libtayo) | |
| Skin | cemiplimab-rwlc | PD-1 |
| | (Libtayo) | |
| Lung | ceritinib (Zykadia) | ALK + IGF-1R + INSR + ROS1 |
| Colorectal | cetuximab (Erbitux) | EGFR |
| Multiple myeloma | ciltacabtagene | BCMA |
| | autoleucel (Carvykti) | |
| Skin | cobimetinib fumarate | MEK1, MEK2 |
| | (Cotellic) | |
| Lymphoma | copanlisib | PI3Kα, PI3Kδ |
| | hydrochloride (Aliqopa) | |
| Lung | crizotinib (Xalkori) | MTH1 |
| Lymphoma | crizotinib (Xalkori) | MTH1 |
| Myofibroblastic | crizotinib (Xalkori) | MTH1 |
| Brain | dabrafenib (Tafinlar) | BRAF, CRAF |
| Lung | dabrafenib mesylate | BRAF, CRAF |
| | (Tafinlar) | |
| Skin | dabrafenib mesylate | BRAF, CRAF |
| | (Tafinlar) | |
| Solid tumors anywhere | dabrafenib mesylate | BRAF, CRAF |
| in the body | (Tafinlar) | |
| Thyroid | dabrafenib mesylate | BRAF, CRAF |
| | (Tafinlar) | |
| Lung | dacomitinib (Vizimpro) | EGFR + EGFR exon 21 L858R mutation + |
| | | EGFR-Ex19del + HER2 + HER4 |
| Multiple myeloma | daratumumab | CD38 + Hyaluronic acid |
| | (Darzalex) | |
| Multiple myeloma | daratumumab and | CD38 + Hyaluronic acid |
| | hyaluronidase-fihj | |
| | (Darzalex Faspro) | |
| Prostate | darolutamide (Nubeqa) | AR |
| Leukemia | dasatinib (Sprycel) | Bcr-Abl + EphA2 + FYN + LCK + |
| | | PDGFRβ + Protein-tyrosine kinases + |
| | | SRC + YES1 + c-Kit |
| Neuroblastoma | dinutuximab (Unituxin) | GD2 |
| Endometrial | dostarlimab-gxly | PD-1 |
| | (Jemperli) | |
| Solid tumors anywhere | dostarlimab-gxly | PD-1 |
| in the body | (Jemperli) | |
| Liver and bile duct | durvalumab (Imfinzi) | PDL1 |
| Lung | durvalumab (Imfinzi) | PDL1 |
| Leukemia | duvelisib (Copiktra) | PI3Kγ + PI3Kδ |
| Breast | elacestrant | Erα/ESR1 |
| | dihydrochloride | |
| | (Orserdu) | |
| Multiple myeloma | elotuzumab (Empliciti) | SLAMF7 |
| Leukemia | enasidenib mesylate | IDH2 |
| | (Idhifa) | |
| Colorectal | encorafenib (Braftovi) | BRAF, BRAF V600E |
| Skin | encorafenib (Braftovi) | BRAF V600E, V600K mutation |
| Bladder | enfortumab vedotin-ejfv | Nectin-4, Tubulins |
| | (Padcev) | |
| Lung | entrectinib (Rozlytrek) | ALK + ROS1 + TRKA + TrkB + TrkC |
| Solid tumors anywhere | entrectinib (Rozlytrek) | ALK + ROS1 + TRKA + TrkB + TrkC |
| in the body | | |
| Prostate | enzalutamide (Xtandi) | AR |
| Bladder | erdafitinib (Balversa) | FGFR1, FGFR2, FGFR3, FGFR4 |
| Lung | erlotinib hydrochloride | EGFR antagonists, EGFR exon 21 L858R |
| | (Tarceva) | mutation inhibitors, EGFR-Ex19del |
| | | inhibitors |
| Pancreatic | erlotinib hydrochloride | EGFR + EGFR exon 21 L858R mutation + |
| | (Tarceva) | EGFR-Ex19del |
| Breast | everolimus (Afinitor) | mTORC1, mTORC2 |
| Kidney | everolimus (Afinitor) | mTORC1, mTORC2 |
| Pancreatic | everolimus (Afinitor) | mTORC1, mTORC2 |
| Breast | exemestane (Aromasin) | Aromatase |
| Breast | fam-trastuzumab | HER2, TOP1 |
| | deruxtecan-nxki | |
| | (Enhertu) | |
| Gastric | fam-trastuzumab | HER2, TOP1 |
| | deruxtecan-nxki | |
| | (Enhertu) | |
| Lung | fam-trastuzumab | HER2, TOP1 |
| | deruxtecan-nxki | |
| | (Enhertu) | |
| Myelodysplastic and | fedratinib hydrochloride | JAK2, FLT3 |
| myeloproliferative | (Inrebic) | |
| disorders | | |
| Breast | fulvestrant (Faslodex) | ER |
| Liver and bile duct | futibatinib (Lytgobi) | FGFRs |
| Lung | gefitinib (Iressa) | EGFR + EGFR exon 21 L858R mutation + |
| | | EGFR-Exon 19 deletion |
| Leukemia | gemtuzumab | CD33, DNA |
| | ozogamicin | |
| | (MYLOTARG) | |
| Leukemia | gilteritinib fumarate | AXL + FLT3 |
| | (Xospata) | |
| Leukemia | glasdegib | SMO |
| | maleate (Daurismo) | |
| Lymphoma | ibritumomab tiuxetan | CD20 |
| | (Zevalin) | |
| Leukemia | ibrutinib (Imbruvica) | BTK |
| Lymphoma | ibrutinib (Imbruvica) | BTK |
| Multiple myeloma | idecabtagene vicleucel | BCMA |
| | (Abecma) | |
| Leukemia | idelalisib (Zydelig) | PI3Kδ |
| Dermatofibrosarcoma | imatinib mesylate | BCR-ABL, PDGFR, C-Kit |
| protuberans | (Gleevec) | |
| Gastrointestinal | imatinib mesylate | BCR-ABL, PDGFR, C-Kit |
| | (Gleevec) | |
| Leukemia | imatinib mesylate | BCR-ABL, PDGFR, C-Kit |
| | (Gleevec) | |
| Myelodysplastic and | imatinib mesylate | BCR-ABL, PDGFR, C-Kit |
| myeloproliferative | (Gleevec) | |
| disorders | | |
| Systemic mastocytosis | imatinib mesylate | BCR-ABL, PDGFR, C-Kit |
| | (Gleevec) | |
| Liver and bile duct | infigratinib phosphate | FGFR1 + FGFR2 + FGFR3 + FGFR4 |
| | (Truseltiq) | |
| Leukemia | inotuzumab ozogamicin | CD22 + DNA |
| | (Besponsa) | |
| Endocrine and | iobenguane I 131 | NET |
| neuroendocrine tumors | (Azedra) | |
| Colorectal | ipilimumab (Yervoy) | CTLA4 |
| Esophageal | ipilimumab (Yervoy) | CTLA4 |
| Kidney | ipilimumab (Yervoy) | CTLA4 |
| Liver and bile duct | ipilimumab (Yervoy) | CTLA4 |
| Lung | ipilimumab (Yervoy) | CTLA4 |
| Malignant | ipilimumab (Yervoy) | CTLA4 |
| mesothelioma | | |
| Skin | ipilimumab (Yervoy) | CTLA4 |
| Multiple myeloma | isatuximab-irfc | CD38 |
| | (Sarclisa) | |
| Leukemia | ivosidenib (Tibsovo) | IDH1 |
| Multiple myeloma | ixazomib citrate | proteasome |
| | (Ninlaro) | |
| Endocrine and | lanreotide acetate | SSTR |
| neuroendocrine tumors | (Somatuline Depot) | |
| Breast | lapatinib ditosylate | EGFR |
| | (Tykerb) | |
| Solid tumors anywhere | larotrectinib sulfate | TRKA + TrkB + TrkC |
| in the body | (Vitrakvi) | |
| Endometrial | lenvatinib mesylate | FGFR1 + FGFR2 + FGFR3 + FGFR4 + |
| | (Lenvima) | PDGFRα + RET + VEGFR1 + VEGFR2 + |
| | | VEGFR3 + c-Kit |
| Kidney | lenvatinib mesylate | FGFR1 + FGFR2 + FGFR3 + FGFR4 + |
| | (Lenvima) | PDGFRα + RET + VEGFR1 + VEGFR2 + |
| | | VEGFR3 + c-Kit |
| Liver and bile duct | lenvatinib mesylate | FGFR1 + FGFR2 + FGFR3 + FGFR4 + |
| | (Lenvima) | PDGFRα + RET + VEGFR1 + VEGFR2 + |
| | | VEGFR3 + c-Kit |
| Thyroid | lenvatinib mesylate | FGFR1 + FGFR2 + FGFR3 + FGFR4 + |
| | (Lenvima) | PDGFRα + RET + VEGFR1 + VEGFR2 + |
| | | VEGFR3 + c-Kit |
| Breast | letrozole | CDK4/ER |
| | (Femara)/Ribociclib | |
| | Succinate | |
| Lymphoma | lisocabtagene | CD19 |
| | maraleucel (Breyanzi) | |
| Lymphoma | loncastuximab tesirine- | CD19 + DNA |
| | lpyl (Zynlonta) | |
| Lung | lorlatinib (Lorbrena) | ALK + ROS1 |
| Prostate | lutetium Lu 177 | PSMA |
| | vipivotide tetraxetan | |
| | (Pluvicto) | |
| Endocrine and | lutetium Lu 177- | SSTR2 |
| neuroendocrine tumors | dotatate (Lutathera) | |
| Breast | margetuximab-cmkb | HER2 |
| | (Margenza) | |
| Leukemia | midostaurin (Rydapt) | FLT3 + PDGFR + PKC + Syk + VEGFR2 + |
| | | c-Kit |
| Systemic mastocytosis | midostaurin (Rydapt) | FLT3 + PDGFR + PKC + Syk + VEGFR2 + |
| | | c-Kit |
| Ovarian epithelial, | mirvetuximab | FOLR1 + Tubulin |
| fallopian tube, and | soravtansine-gynx | |
| primary peritoneal | (Elahere) | |
| Lung | mobocertinib succinate | EGFR exon 20 + HER2 exon 20 |
| | (Exkivity) | |
| Lymphoma | mogamulizumab-kpkc | CCR4 |
| | (Poteligeo) | |
| Lymphoma | mosunetuzumab-axgb | CD20, CD3 |
| | (Lunsumio) | |
| Leukemia | moxetumomab | CD22 |
| | pasudotox- | |
| | tdfk (Lumoxiti) | |
| Neuroblastoma | naxitamab-gqgk | GD2 |
| | (Danyelza) | |
| Lung | necitumumab | EGFR |
| | (Portrazza) | |
| Breast | neratinib maleate | EGFR, HER2, HER4 |
| | (Nerlynx) | |
| Leukemia | nilotinib (Tasigna) | Bcr-Abl + CSF-1R + DDR1 + PDGFR + |
| | | c-Kit |
| Ovarian epithelial, | niraparib tosylate | PARP1 + PARP2 |
| fallopian tube, and | monohydrate (Zejula) | |
| primary peritoneal | | |
| Bladder | nivolumab (Opdivo) | PD-1 |
| Colorectal | nivolumab (Opdivo) | PD-1 |
| Esophageal | nivolumab (Opdivo) | PD-1 |
| Kidney | nivolumab (Opdivo) | PD-1 |
| Liver and bile duct | nivolumab (Opdivo) | PD-1 |
| Lung | nivolumab (Opdivo) | PD-1 |
| Lymphoma | nivolumab (Opdivo) | PD-1 |
| Malignant | nivolumab (Opdivo) | PD-1 |
| mesothelioma | | |
| Skin | nivolumab (Opdivo) | PD-1 |
| Stomach (gastric) | nivolumab (Opdivo) | PD-1 |
| Skin | nivolumab and | LAG3 + PD-1 |
| | relatlimab-rmbw | |
| | (Opdualag) | |
| Leukemia | obinutuzumab (Gazyva) | CD20 |
| Lymphoma | obinutuzumab (Gazyva) | CD20 |
| Leukemia | ofatumumab (Arzerra) | CD20 |
| Ovarian epithelial, | olaparib (Lynparza) | PARP1, PARP2, PARP3 |
| fallopian tube, and | | |
| primary peritoneal | | |
| Breast | olaparib (Lynparza) | PARP1, PARP2, PARP3 |
| Pancreatic | olaparib (Lynparza) | PARP1, PARP2, PARP3 |
| Prostate | olaparib (Lynparza) | PARP1, PARP2, PARP3 |
| Leukemia | olutasidenib (Rezlidhia) | IDH1 |
| Lung | osimertinib mesylate | EGFR + EGFR T790M + EGFR exon 21 |
| | (Tagrisso) | L858R mutation + EGFR-Ex19del |
| Myelodysplastic and | pacritinib citrate | CSF-1R + FLT3 + IRAK1 + JAK2 |
| myeloproliferative | (Vonjo) | |
| disorders | | |
| Breast | palbociclib (Ibrance) | CDK4, CDK6 |
| Colorectal | panitumumab | EGFR |
| | (Vectibix) | |
| Kidney | pazopanib | FGFR1 + FGFR3 + Flt3L + ITK + LCK + |
| | hydrochloride (Votrient) | PDGFRα + PDGFRβ + VEGFR1 + |
| | | VEGFR2 + VEGFR3 + c-Kit |
| Soft tissue sarcoma | pazopanib | FGFR1 + FGFR3 + Flt3L + ITK + LCK + |
| | hydrochloride (Votrient) | PDGFRα + PDGFRβ + VEGFR1 + |
| | | VEGFR2 + VEGFR3 + c-Kit |
| Breast | pembrolizumab | PD-1 |
| | (Keytruda) | |
| Cervical | pembrolizumab | PD-1 |
| | (Keytruda) | |
| Colorectal | pembrolizumab | PD-1 |
| | (Keytruda) | |
| Endometrial | pembrolizumab | PD-1 |
| | (Keytruda) | |
| Esophageal | pembrolizumab | PD-1 |
| | (Keytruda) | |
| Kidney | pembrolizumab | PD-1 |
| | (Keytruda) | |
| Liver and bile duct | pembrolizumab | PD-1 |
| | (Keytruda) | |
| Lung | pembrolizumab | PD-1 |
| | (Keytruda) | |
| Lymphoma | pembrolizumab | PD-1 |
| | (Keytruda) | |
| Skin | pembrolizumab | PD-1 |
| | (Keytruda) | |
| Solid tumors anywhere | pembrolizumab | PD-1 |
| in the body | (Keytruda) | |
| Stomach (gastric) | pembrolizumab | PD-1 |
| | (Keytruda) | |
| Leukemia | pemigatinib (Pemazyre) | FGFR1, FGFR2, FGFR3, FGFR4 |
| Liver and bile duct | pemigatinib (Pemazyre) | FGFR1, FGFR2, FGFR3, FGFR4 |
| Lymphoma | pemigatinib (Pemazyre) | FGFR1, FGFR2, FGFR3, FGFR4 |
| Myelodysplastic and | pemigatinib (Pemazyre) | FGFR1, FGFR2, FGFR3, FGFR4 |
| myeloproliferative | | |
| disorders | | |
| Breast | pertuzumab (Perjeta) | HER2 |
| Breast | pertuzumab, | HER2, Hyaluronic acid |
| | trastuzumab, and | |
| | hyaluronidase-zzxf | |
| | (Phesgo) | |
| Giant cell tumor | pexidartinib | CSF-1R + FLT3 + c-Kit |
| | hydrochloride (Turalio) | |
| Lymphoma | pirtobrutinib (Jaypirca) | BTK C481S |
| Lymphoma | polatuzumab vedotin- | CD79B + Tubulin |
| | piiq (Polivy) | |
| Leukemia | ponatinib hydrochloride | Bcr-Abl + Protein-tyrosine kinases |
| | (Iclusig) | |
| Lymphoma | pralatrexate (Folotyn) | Bcr-Abl + Protein-tyrosine kinases |
| Lung | pralsetinib (Gavreto) | RET |
| Thyroid | pralsetinib (Gavreto) | RET |
| Prostate | radium 223 dichloride | DNA |
| | (Xofigo) | |
| Colorectal | ramucirumab (Cyramza) | VEGFR2 |
| Gastric | ramucirumab (Cyramza) | VEGFR2 |
| Liver and bile duct | ramucirumab (Cyramza) | VEGFR2 |
| Lung | ramucirumab (Cyramza) | VEGFR2 |
| Stomach (gastric) | ramucirumab (Cyramza) | VEGFR2 |
| Colorectal | regorafenib (Stivarga) | Abl family, BRAF, BRAF V600E, CRAF, |
| | | CSF-1R, DDR2, EphA2, FGFR1, FGFR2, |
| | | FRK, MAPK11, PDGFRa, PDGFRb, |
| | | RET, TRKA, Tie-2, VEGFR1, VEGFR2, |
| | | VEGFR3, c-kit |
| Gastrointestinal | regorafenib (Stivarga) | Abl family, BRAF, BRAF V600E, CRAF, |
| | | CSF-1R, DDR2, EphA2, FGFR1, FGFR2, |
| | | FRK, MAPK11, PDGFRa, PDGFRb, |
| | | RET, TRKA, Tie-2, VEGFR1, VEGFR2, |
| | | VEGFR3, c-kit |
| Liver and bile duct | regorafenib (Stivarga) | Abl family, BRAF, BRAF V600E, CRAF, |
| | | CSF-1R, DDR2, EphA2, FGFR1, FGFR2, |
| | | FRK, MAPK11, PDGFRa, PDGFRb, |
| | | RET, TRKA, Tie-2, VEGFR1, VEGFR2, |
| | | VEGFR3, c-kit |
| Skin | retifanlimab-dlwr | PD-1 |
| | (Zynyz) | |
| Breast | ribociclib (Kisqali) | CDK4, CDK6 |
| Gastrointestinal | ripretinib (Qinlock) | EGFR + PDGFRα + c-Kit |
| Leukemia | rituximab (Rituxan) | CD20 + Hyaluronic acid |
| Lymphoma | rituximab (Rituxan) | CD20 + Hyaluronic acid |
| Leukemia | rituximab and | CD20 + Hyaluronic acid |
| | hyaluronidase human | |
| | (Rituxan Hycela) | |
| Lymphoma | rituximab and | CD20 + Hyaluronic acid |
| | hyaluronidase human | |
| | (Rituxan Hycela) | |
| Lymphoma | romidepsin (Istodax) | HDAC |
| Ovarian epithelial, | rucaparib | PARP1 + PARP2 + PARP3 |
| fallopian tube, and | camsylate (Rubraca) | |
| primary peritoneal | | |
| Prostate | rucaparib camsylate | PARP1 + PARP2 + PARP3 |
| | (Rubraca) | |
| Myelodysplastic and | ruxolitinib phosphate | JAK1 + JAK2 |
| myeloproliferative | (Jakafi) | |
| disorders | | |
| Breast | sacituzumab govitecan- | TOP1 |
| | hziy (Trodelvy) | |
| Lymphoma | selinexor (Xpovio) | XPO1 |
| Multiple myeloma | selinexor (Xpovio) | XPO1 |
| Lung | selpercatinib (Retevmo) | RET |
| Solid tumors anywhere | selpercatinib (Retevmo) | RET |
| in the body | | |
| Thyroid | selpercatinib (Retevmo) | RET |
| Plexiform | selumetinib sulfate | MEK1 + MEK2 |
| neurofibroma | (Koselugo) | |
| Soft tissue sarcoma | sirolimus protein-bound | MUT + mTOR |
| | particles (Fyarro) | |
| Skin | sonidegib (Odomzo) | SMO |
| Kidney | sorafenib tosylate | BRAF inhibitors, CRAF inhibitors, |
| | (Nexavar) | FLT3 + PDGFRβ + RET + VEGFR1 + |
| | | VEGFR2 + VEGFR3 + c-Kit |
| Liver and bile duct | sorafenib tosylate | BRAF + CRAF + FLT3 + PDGFRβ + RET + |
| | (Nexavar) | VEGFR1 + VEGFR2 + VEGFR3 + c-Kit |
| Thyroid | sorafenib tosylate | BRAF + CRAF + FLT3 + PDGFRβ + RET + |
| | (Nexavar) | VEGFR1 + VEGFR2 + VEGFR3 + c-Kit |
| Lung | sotorasib (Lumakras) | KRAS G12C |
| Gastrointestinal | sunitinib malate | PDGFR + RTK + VEGFR |
| | (Sutent) | |
| Kidney | sunitinib malate | PDGFR + RTK + VEGFR |
| | (Sutent) | |
| Pancreatic | sunitinib malate | PDGFR + RTK + VEGFR |
| | (Sutent) | |
| Lymphoma | tafasitamab-cxix | CD19 |
| | (Monjuvi) | |
| Leukemia | tagraxofusp-erzs | CD123 |
| | (Elzonris) | |
| Breast | talazoparib tosylate | PARP1, PARP2 |
| | (Talzenna) | |
| Breast | tamoxifen citrate | ER |
| | (Soltamox) | |
| Lymphoma | tazemetostat | EZH2 |
| | hydrobromide | |
| | (Tazverik) | |
| Soft tissue sarcoma | tazemetostat | EZH2 |
| | hydrobromide | |
| | (Tazverik) | |
| Skin | tebentafusp-tebn | CD3 + gp100 |
| | (Kimmtrak) | |
| Multiple myeloma | teclistamab-cqyv | CD3 + gp100 |
| | (Tecvayli) | |
| Kidney | temsirolimus (Torisel) | CD3 + gp100 |
| Lung | tepotinib hydrochloride | MET |
| | (Tepmetko) | |
| Leukemia | tisagenlecleucel | CD19 |
| | (Kymriah) | |
| Lymphoma | tisagenlecleucel | CD19 |
| | (Kymriah) | |
| Cervical | tisotumab vedotin-tftv | Tubulin, tissue factor |
| | (Tivdak) | |
| Kidney | tivozanib hydrochloride | (VEGFR)-1, VEGFR-2 and VEGFR-3, |
| | (Fotivda) | c-kit, and PDGFR β |
| Breast | toremifene (Fareston) | ER |
| Brain | trametinib (Mekinist) | MEK1 and MEK2 |
| Lung | trametinib dimethyl | MEK1 and MEK2 |
| | sulfoxide (Mekinist) | |
| Skin | trametinib dimethyl | MEK1 and MEK2 |
| | sulfoxide (Mekinist) | |
| Solid tumors anywhere | trametinib dimethyl | MEK1 and MEK2 |
| in the body | sulfoxide (Mekinist) | |
| Thyroid | trametinib dimethyl | MEK1 and MEK2 |
| | sulfoxide (Mekinist) | |
| Breast | trastuzumab (Herceptin) | HER2 |
| Esophageal | trastuzumab (Herceptin) | HER2 |
| Stomach (gastric) | trastuzumab (Herceptin) | HER2 |
| Liver and bile duct | tremelimumab-actl | CTLA-4, CD80, and CD86 |
| | (Imjudo) | |
| Lung | tremelimumab-actl | CTLA-4, CD80, and CD86 |
| | (Imjudo) | |
| Breast | tucatinib (Tukysa) | HER2 |
| Colorectal | tucatinib (Tukysa) | HER2 |
| Thyroid | vandetanib (Caprelsa) | VEGFR-2, EGFR, RET |
| Skin | vemurafenib (Zelboraf) | BRAF, BRAF V600E, CRAF, ARAF, |
| | | SRMS, ACK1, MAP4K5, FGR |
| Leukemia | venetoclax (Venclexta) | BCL-2 |
| Lymphoma | venetoclax (Venclexta) | BCL-2 |
| Lymphoma | vorinostat (Zolinza) | HDAC1, HDAC2 and HDAC3 (Class I) |
| | | and HDAC6 (Class II) |
| Leukemia | zanubrutinib (Brukinsa) | BTK |
| Lymphoma | zanubrutinib (Brukinsa) | BTK |
| Colorectal | ziv-aflibercept (Zaltrap) | PGF, VEGF-A |

The methods provided herein provide a deeper understanding of the changes in proteins that are associated or cause diseases such as cancer, allowing the identification of biomarkers and design of treatments that target these proteins. In some embodiments, the biomarker may include an epigenetic signature, such as a methylation state, methylation score and/or DNA fragmentation pattern/score. In some embodiments, the epigenetic signature can be determined for one or more regions that include, but not limited to, transcription start sites, promoter regions, CTCF binding regions and regulatory protein binding regions. In some embodiments, the epigenetic signature is determined for one or more regions that include, but not limited to, transcription start sites, promoter regions, intergenic regions and/or intronic regions associated with at least one or more disease-related gene. Such treatments may include small-molecule drugs or monoclonal antibodies. The methods may also improve biomarker testing in individuals suffering from disease and help determine if the individual is a candidate for a certain drug or combination of drugs based on the presence or absence of the biomarker. Additionally, the methods can improve identification of mutations that contribute to the development of resistance to targeted therapy. Consequently, the analysis techniques may reduce unnecessary or untimely therapeutic interventions, patient suffering, and patient mortality.

### EXAMPLES

The present disclosure is also described and demonstrated by way of the following examples. However, the use of these and other examples anywhere in the specification is illustrative only and in no way limits the scope and meaning of a claimed invention or of any exemplified term. Likewise, a claimed invention is not limited to any preferred embodiment described herein. Indeed, many modifications and variations of a claimed invention may be apparent to those skilled in the art upon reading this specification, and such variations can be made without departing from that claimed invention in spirit or in scope. A claimed invention is therefore to be limited only by the terms of the appended claims along with the full scope of equivalents to which those claims are entitled.

### Example 1

The standard NGS-based multiplex immunoassay workflow utilizing Proximity Extension Assay techniques (“PEA”) is inefficient and has high sequencing costs because it is limited in the number of unique DNA-encoded protein barcodes. Given that there are more immunoassay targets-currently 1536 proteins, soon to be expanded to 3072- than DNA-encoded protein barcodes (384), barcodes must be re-used in the standard assay, and targets with the same barcode must be processed separately all the way through physically resolved NGS. For the 3072 target immunoassay, 8 protein panels each with 384 non-overlapping protein barcodes are processed in parallel. In sequencing, each panel can be run on a separate flow cell (e.g. 8 NextSeq550 flowcells) or on separate, physically addressable lanes on the same flowcell (e.g. 4 NovaSeq SP flowcells, using the XP individual lane loading workflow). For convenience, sequencer instrument efficiency and cost savings it would be advantageous to be able to combine all the panels into one NGS pool to load on single (physically addressable) flowcell.

A solution to the problem above is to add a ‘panel barcoding’ step to the PEA NGS library prep workflow. By doing this, the combination of panel barcode and protein barcode on a NGS library molecule (and NGS read) will uniquely specify the protein analyte being detected through NGS. The PEA workflow specifies the protein barcode at the initial PEA immunoassay step (shown in the figures), while the sample barcode is encoded during the second PCR, universally tagging all NGS library molecules of a given panel, for a specific sample. The resultant NGS library molecules have the sample and protein barcodes internal to the Illumina sequencing primers and are thus read ‘in-line’. As such, the standard Illumina index reads are not used in this workflow and are available to use to ascribe ‘panel’ barcodes. This can be achieved by adding a PCR step with standard index primers to each sub pool-libraries from different samples, assaying the same protein panel pool. This modification will also allow the ‘sample indexing’ primers for the 2^{nd} PCR to use shorter and more efficient primers. Currently, these primers contain NGS flow cell primers (P5/P7), sequencing read primers, sample index and a hybridization sequence, universal to all the PEA products after first amplification. By having another subsequent step for panel barcoding, sample index primers can be reduced from ˜80nt to ˜50nt. This should make the sample indexing PCR more efficient.

The ‘panel’ barcoding could alternatively be introduced at the 2^{nd} PCR step, the current ‘sample’ barcoding step. The benefit of this would not be needing to add another PCR step which may require some optimization. Additional modifications, such as specific sample-barcode primers for each panel in the 2^{nd} PCR may be implemented. This process could be used for scaling up the assay. The utility of this strategy will become more apparent as the NGS-based immunoassay technology scales to be able to identify more targets.

1. A method for detecting a polypeptide in a plurality of biological samples, comprising:
(a) contacting a first sub-sample of each biological sample with a first panel of probes and contacting a second sub-sample of each biological sample with a second panel of probes, wherein each probe comprises a polypeptide binding domain that specifically recognizes a polypeptide target and a polynucleotide domain that includes at least one target barcode and at least one hybridization domain;
(b) enabling the probes to bind to said polypeptide target;
(c) enabling the at least one hybridization domain to hybridize and form a polynucleotide duplex;
(d) amplifying said polynucleotide duplex, comprising said target barcodes, and generating a population of polynucleotide tags;
(e) modifying said polynucleotide tags by:
1) incubating said population of polynucleotide tags with extension primers capable of hybridizing to the polynucleotide tags and of incorporating at least one additional barcode into said polynucleotide tag,
2) amplifying said population of polynucleotide tags to incorporate the at least one additional barcode;
(f) repeating said modifying; and
(g) identifying the polypeptide using said modified polynucleotide tag,
wherein the at least one additional barcode of step (e) is a sample barcode and the at least one additional barcode of step (f) is a panel barcode.

2. The method of claim 1, wherein the method comprises pooling, for each panel, the modified polynucleotide tags and step (f) is performed on the panel pools.

3. (canceled)

4. The method of claim 1, comprising pooling the modified polynucleotide tags after step (f) and prior to step (g).

5. The method of claim 1, comprising one or more further sub-samples, wherein the one or more further sub-samples are contacted with one or more further panels of probes; and polynucleotide tags derived from the one or more further sub-samples, or pools thereof, are modified with one or more further panel barcodes in step (f).

6. The method of claim 1, wherein the panels comprise the same set of target barcodes.

7. The method of claim 1, wherein the biological samples are bodily fluid samples or derived from bodily fluid samples, for example wherein the bodily fluid is blood.

8. The method of claim 1, wherein each panel of probes comprises, for each polypeptide target, a forward probe and a reverse probe, and wherein the at least one hybridization domain of the forward probe is complementary to the at least one hybridization domain of the reverse probe.

9. The method of claim 1, wherein the identifying is by DNA sequencing.

10. A method for detecting a polypeptide in a biological sample, comprising:
(a) contacting said biological sample with at least one set of probes, wherein each probe comprises a polypeptide binding domain that specifically recognizes a polypeptide target and a polynucleotide domain that includes at least one unique barcode and at least one hybridization domain;
(b) enabling each at least one set of probes to bind to each said polypeptide target;
(c) enabling the at least one hybridization domain to hybridize and form a polynucleotide duplex;
(d) amplifying said polynucleotide duplex, comprising said unique barcodes, and generating a population of polynucleotide tags;
(e) modifying said polynucleotide tags by:
1) incubating said population of polynucleotide tags with extension primers capable of hybridizing and catalyzing extension of the polynucleotide tags and of incorporating at least one additional unique barcode into said polynucleotide tag;
2) amplifying said population of polynucleotide tags to incorporate the at least one additional unique barcode; and
(f) identifying the polypeptide using said modified polynucleotide tag.

11. The method of claim 10, further comprising pooling together a population of modified polynucleotide tags prior to said identifying, wherein said pooled population includes modified polynucleotide tags from different biological samples, from different polypeptide panels, or from different experimental conditions.

12. The method of claim 10, wherein said modifying is repeated more than once prior to said identifying.

13. The method of claim 10, wherein said at least one hybridization domain on said polynucleotide domain is complementary to at least one hybridization domain on the same polynucleotide.

14. The method of claim 10, wherein said population of polynucleotide tags is divided into separate aliquots prior to said amplifying of said polynucleotide duplex.

15. The method of claim 10, wherein said amplifying comprises a polymerase chain reaction or a quantitative polymerase chain reaction.

16. The method of claim 10, wherein the identifying is performed by analyzing sequencing data from a sequencer.

17.-18. (canceled)

19. A method for detecting a molecule in a biological sample, comprising:
(a) contacting said biological sample with at least one probe, wherein each probe comprises a polypeptide binding domain that specifically recognizes a target on said polypeptide and a polynucleotide domain that includes at least one unique barcode and at least one hybridization domain;
(b) enabling each at least one probe to bind to each said polypeptide target;
(c) enabling the at least one hybridization domain to hybridize and form a polynucleotide duplex;
(d) amplifying said polynucleotide duplex, comprising said unique barcodes, and generating a population of polynucleotide tags;
(e) modifying said polynucleotide tags by:
1) incubating said population of polynucleotide tags with extension primers capable of hybridizing and catalyzing extension of the polynucleotide tags and of incorporating at least one additional unique barcode into said polynucleotide tag;
2) amplifying said population of polynucleotide tags to incorporate the at least one additional unique barcode; and
(f) identifying the molecule using said modified polynucleotide tag.

20. The method of claim 18, wherein said molecule in a biological sample is selected from the group consisting of: proteins, polynucleotides, and protein complexes.

21. The method of claim 19, wherein said molecule in a biological sample is selected from the group consisting of: proteins, polynucleotides, and protein complexes.

22. The method of claim 20, further comprising pooling together a population of modified polynucleotide tags prior to said identifying, wherein said pooled population includes modified polynucleotide tags from different biological samples, from different polypeptide panels, or from different experimental conditions.

23. The method of claim 21, further comprising pooling together a population of modified polynucleotide tags prior to said identifying, wherein said pooled population includes modified polynucleotide tags from different biological samples, from different polypeptide panels, or from different experimental conditions.

24.-32. (canceled)
